(19) United States
(12) Patent Application Publication
(10) Pub. No.: US 2019/0105514 A1
(43) Pub. Date: Apr. 11, 2019

(54) OPTICAL SYSTEM FOR RADIATION TREATMENT

(71) Applicants: Varian Medical Systems, Inc., Palo Alto, CA (US); Varian Medical Systems International AG, Cham (CH)

(72) Inventors: Martin Amstutz, Doettingen (CH); Patrik Kunz, Baden (CH); Stefan G. Scheib, Wadenswil (CH); Roland Meier, Baden-Daettwil (CH); Fergus Quigley, Seattle, WA (US); Eric Hadford, Snohomish, WA (US); Ron Weaver, Sammamish, WA (US); Roland Perez, Woodinville, WA (US); Sun-Kai Lin, Seattle, WA (US)

(21) Appl. No.: 15/728,475

(22) Filed: Oct. 9, 2017

(51) Int. Cl. A61N 5/10; A61B 5/11; A61B 5/113; A61B 90/00; A61B 34/20

(52) U.S. Cl. CPC A61N 5/1049; A61B 5/1127; A61B 5/113; A61B 2034/2057; A61B 34/20; A61B 2090/397; A61B 90/39

(57) ABSTRACT

An apparatus includes: a structure for coupling to a patient; a plurality of reflective markers coupled to the structure, wherein the reflective markers are configured to reflect non-visible light; and a securing mechanism configured to secure the structure relative to the patient. An apparatus includes: a first light source configured to project a first structured light onto an object, wherein the first structured light is non-visible; a first camera configured to obtain a first image of the first structured light as projected onto the object; a second camera configured to obtain a second image of the first structured light as projected onto the object; and a processing unit configured to process the first and second images from the first camera and the second camera; wherein the first camera and the second camera are configured for non-visible light detection.

### FIELD

The field relates to optical systems for use in medical processes, and more particularly, to optical systems and methods for use in radiation treatment.

### BACKGROUND

Radiation therapy involves medical procedures that selectively expose certain areas of a human body, such as cancerous tumors, to doses of radiation. The purpose of the radiation therapy is to irradiate the targeted biological tissue such that undesirable tissue is destroyed. Radiation has also been-used to obtain image of tissue for diagnostic or treatment purposes.

During delivery of radiation towards a patient, it may be desirable to ensure that a patient remains at a certain position. Also, it may be desirable to know the position of a patient and/or the position of various components of the treatment system during the treatment session in order to prevent collision between the patient and the components.

### SUMMARY

An apparatus for use in a medical procedure includes: a structure for coupling to a patient; a plurality of reflective markers coupled to the structure, wherein the reflective markers are configured to reflect non-visible light; and a securing mechanism configured to secure the structure relative to the patient.

Optionally, the structure comprises a frame, and a first base coupled to the frame.

Optionally, the securing mechanism comprise a first adhesive for attachment to the patient.

Optionally, the apparatus further includes a nose piece coupled to the frame.

Optionally, the nose piece comprises an adhesive for attachment to the patient.

Optionally, the frame comprises a laser alignment mark.

Optionally, the plurality of reflective markers comprises four reflective markers.

Optionally, the apparatus further includes a second base having a second adhesive for attachment to the patient, wherein the second base is coupled to the frame.

Optionally, the first base is rotatably coupled to the frame.

Optionally, the first base is rotatably coupled to the frame via a ball and socket connector.

Optionally, the first base is configured to detachably couple to the frame.

Optionally, the first base is configured to detachably couple to the frame via a snap-connector.

Optionally, the structure is configured for coupling to a head of the patient.

Optionally, the structure is configured for coupling to a torso of the patient.

Optionally, the structure is configured for coupling to a limb of the patient.

Optionally, the structure comprises a frame and the securing mechanism comprises a face mask coupled to the frame.

Optionally, the structure is configured for coupling to a skin above an ear of the patient.

Optionally, the structure comprises eyewear.

Optionally, the structure comprises a strap for placement around a head of the patient.

Optionally, the securing mechanism comprises a strap connector or a strap tightening knob.

Optionally, the structure comprises a mouth piece.

Optionally, the structure comprises a hat.

Optionally, the hat comprises a first portion made from a first material, and a second portion made from a second material, and wherein the first material is more elastic than the second material.

Optionally, the apparatus further includes one or more cameras for viewing the plurality of reflective markers, and one or more light sources for providing structured light for surface scanning.

An apparatus for use in a medical procedure includes: a structure for coupling to a patient support; a plurality of reflective markers coupled to the structure, wherein the reflective markers are configured to reflect non-visible light; and a securing mechanism configured to secure the structure relative to the patient support.

Optionally, the structure comprises a first arm, and a second arm moveably coupled to the first arm.

An apparatus for use in a medical procedure includes: a first light source configured to project a first structured light onto an object, wherein the first structured light is non-visible; a first camera configured to obtain a first image of the first structured light as projected onto the object; a second camera configured to obtain a second image of the first structured light as projected onto the object; and a processing unit configured to process the first and second images from the first camera and the second camera; wherein the first camera and the second camera are configured for non-visible light detection. Optionally, the apparatus may further include a third camera.

Optionally, the apparatus further includes a first time-of-flight camera configured to obtain a first depth image of the object.

Optionally, the apparatus further includes a second time-of-flight camera configured to obtain a second depth image of the object.

Optionally, the apparatus further includes a frame, wherein the first light source, the first camera, and the second camera are coupled to the frame.

Optionally, the first camera is moveably mounted to the frame, and the second camera is moveably mounted to the frame.

Optionally, the processing unit is configured to perform imaging processing based on input from the first camera and the second camera for patient setup, patient monitoring, device monitoring, collision prevention, respiratory motion control, or any combination of the foregoing.

Optionally, the processing unit is configured to determine a surface based on input from the first camera and the second camera.

Optionally, the surface corresponds with an abdominal region of a patient, and/or a chest of the patient, and wherein the processing unit is further configured to correlate a position of the surface with an interior region of the patient.

Other and further aspects and features will be evident from reading the following detailed description.

### BRIEF DESCRIPTION OF THE DRAWINGS

The drawings illustrate the design and utility of embodiments, in which similar elements are referred to by common reference numerals. In order to better appreciate how advantages and objects are obtained, a more particular description of the embodiments will be described with reference to the accompanying drawings. Understanding that these drawings depict only exemplary embodiments and are not therefore to be considered limiting in the scope of the claimed invention.

FIG. 1 illustrates a treatment system that includes an optical system;

FIG. 2 illustrates another optical system;

FIG. 3A illustrates an apparatus having markers;

FIG. 3B illustrates another apparatus having markers;

FIG. 3C illustrates another apparatus having markers;

FIG. 3D illustrates another apparatus having markers;

FIG. 3E illustrates another apparatus having markers;

FIG. 3F illustrates another apparatus having markers;

FIG. 3G illustrates another apparatus having markers;

FIG. 3H illustrates another apparatus having markers;

FIG. 3I illustrates another apparatus having markers;

FIG. 3J illustrates another apparatus having markers;

FIG. 3K illustrates another apparatus having markers;

FIG. 3L illustrates another apparatus having markers;

FIG. 4 illustrates another apparatus having markers;

FIG. 5 illustrates a method; and

FIG. 6 is a diagram of a processing system with which embodiments described herein may be implemented.

### DESCRIPTION OF THE EMBODIMENTS

Various embodiments are described hereinafter with reference to the figures. It should be noted that the figures may or may not be drawn to scale and that elements of similar structures or functions are represented by like reference numerals throughout the figures. It should also be noted that the figures are only intended to facilitate the description of the embodiments. They are not intended as an exhaustive description of the claimed invention or as a limitation on the scope of the claimed invention. In addition, an illustrated embodiment needs not have all the aspects or advantages of the invention shown. An aspect or an advantage described in conjunction with a particular embodiment is not necessarily limited to that embodiment and can be practiced in any other embodiments even if not so illustrated or if not so explicitly described.

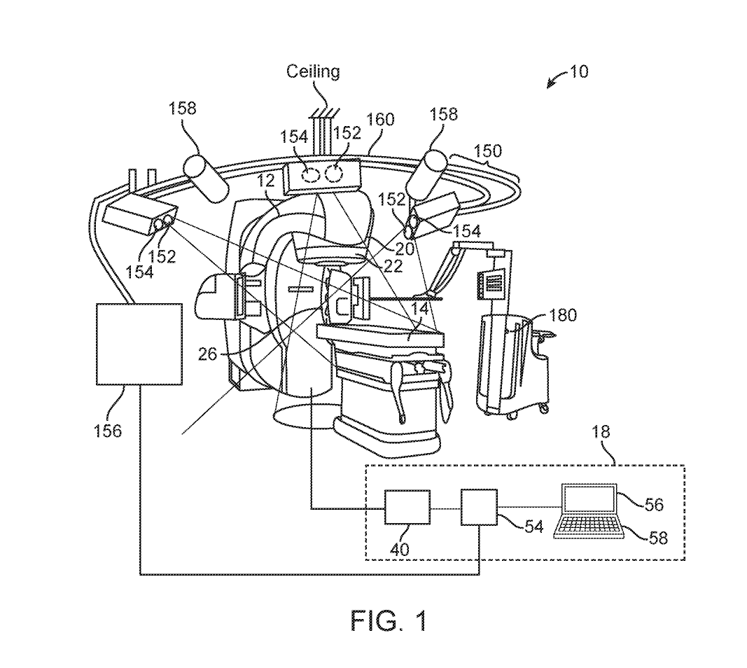

FIG. 1 illustrates a medical system 10. The medical system 10 is a treatment system that includes a gantry 12, a patient support 14 for supporting a patient (not shown), and a control system 18 for controlling an operation of the gantry 12. The gantry 12 is in a form of an arm, but in other embodiments, the gantry 12 may have other forms (such as a ring form, etc.). The system 10 also includes a radiation source 20 that projects a beam 26 of radiation towards a patient while the patient is supported on support 14, and a collimator system 22 for controlling a delivery of the radiation beam 26. The collimator may be configured to adjust a cross sectional shape of the beam 26. The radiation source 20 can be configured to generate a cone beam, a fan beam, or other types of radiation beams in different embodiments.

In some cases, the system 10 may include an imager located at an operative position relative to the source 20 (e.g., under the support 14). In the illustrated embodiments, the radiation source 20 is a treatment radiation source for providing treatment energy. In such cases, the treatment energy may be used to obtain images. In order to obtain imaging using treatment energies, the imager is configured to generate images in response to radiation having treatment energies (e.g., MV imager). In other embodiments, in addition to being a treatment radiation source, the radiation source 20 can also be a diagnostic radiation source for providing diagnostic energy for imaging purpose. In further embodiments, the system may include the radiation source 20 for providing treatment energy, and one or more other radiation sources for providing diagnostic energy. In some embodiments, the treatment energy is generally those energies of 160 kilo-electron-volts (keV) or greater, and more typically 1 mega-electron-volts (MeV) or greater, and diagnostic energy is generally those energies below the high energy range, and more typically below 160 keV. In other embodiments, the treatment energy and the diagnostic energy can have other energy levels, and refer to energies that are used for treatment and diagnostic purposes, respectively. In some embodiments, the radiation source 20 is able to generate X-ray radiation at a plurality of photon energy levels within a range anywhere between approximately 10 keV and approximately 20 MeV. In other embodiments, the radiation source 20 may be configured to generate radiation at other energy ranges.

In the illustrated embodiments, the control system 18 includes a processing unit 54, such as a computer processor, coupled to a control 40. The control system 18 may also include a monitor 56 for displaying data and an input device 58, such as a keyboard or a mouse, for inputting data. The operation of the radiation source 20 and the gantry 12 are controlled by the control 40, which provides power and timing signals to the radiation source 20, and controls a rotational speed and position of the gantry 12, based on signals received from the processor 54. In some cases, the control 40 may also control the collimator system 22 and the position of the patient support 14. In addition, in some cases, the control 40 may be configured to control the beam 26 (e.g., beam hold for gating). Furthermore, the control 40 may be configured to control an imaging process (e.g., triggering of imaging). Although the control 40 is shown as a separate component from the gantry 12 and the processor 54, in alternative embodiments, the control 40 can be a part of the gantry 12 or the processing unit 54.

As shown in FIG. 1, the system 10 also includes an optical system 150. The optical system 150 includes a light source 152, multiple cameras 154, and a processing unit 156 in communication with the cameras 154. In the illustrated example, the light source 152 is configured to provide structured light and/or non-structured light. Also, as shown in the figure, the optical system 150 has three cameras 154. In other embodiments, the optical system 150 may have fewer than three cameras 154 (e.g., one camera 154 or two cameras), or more than three cameras 154. Also, in other embodiments, the optical system 150 may include multiple light sources 152.

Also, in some embodiments, the structured light and/or non-structured light provided by the light source 152 may be in an infrared range (e.g., having infrared wavelength(s)). This technique obviates the need to use very intense light source(s), which may “blind” the patient, particularly during head, neck, and breast treatments in which the light is directed towards the upper part of the patient. In other embodiments, the light source 152 may be configured to provide non-visible light having other wavelengths (e.g., ultraviolet light). Also, use of non-visible light is advantageous because unlike video-based system that uses visible wavelengths, it does not exhibit stroboscopic effects that may confuse the patient, and it does not trigger symptoms of motion thickness.

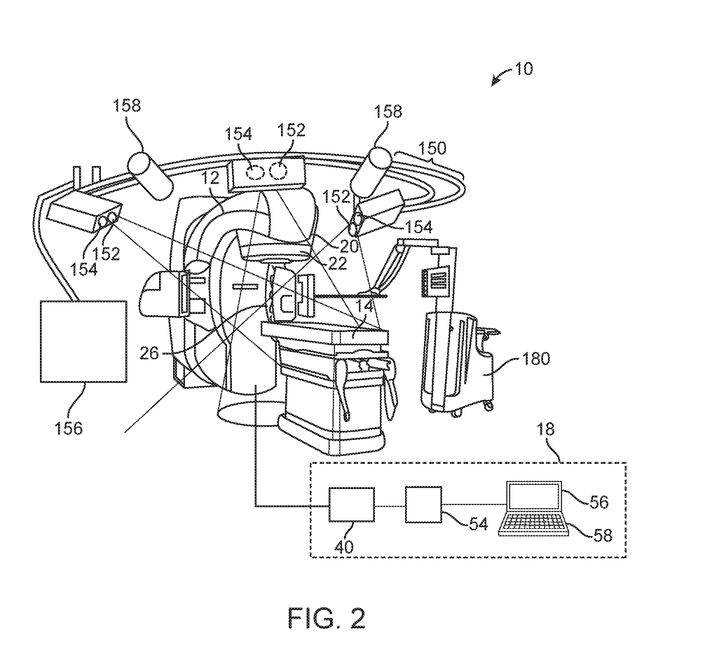

The optical system 150 may also optionally include a frame 160 to which the cameras 154 and the light source 152 may be mounted. The frame 160 may be mounted to a ceiling and/or a wall of a room in which the treatment system 10 is located. Alternatively, the frame 160 may be mounted to the treatment system 10 (FIG. 2). The cameras 154 with the frame 160 may be preassembled at a factory, which allows easy installation at the medical facility. The cameras 154 may be moveably mounted to the frame 160. In one implementation, each of the cameras 154 may be rotatably mounted to the frame 160 (e.g., via a ball joint) so that the camera 154 is rotatable about one or more axes with respect to the frame 160. Similarly, the light source 152 may be moveably mounted to the frame 160. For example, the light source 152 may be rotatably mounted to the frame 160 (e.g., via a ball joint) so that the light source 152 is rotatable about one or more axes with respect to the frame 160. In other embodiments, instead of ball joints, the cameras 154 and the light source 152 may be moveably mounted to the frame 160 using other connectors, such as arms, so that the cameras 154 and the light source 152 are moveable with respect to the frame 160. In other embodiments, the one or more of the cameras 154 and/or the light source 152 may be mounted directly to the treatment system 10 or a room.

Furthermore, in other embodiments, instead of having only one light source 152, the optical system 150 may include multiple light sources 152. In some embodiments, each of the light sources 152 may be configured to provide structured light and non-structured light. In other embodiments, one or more of the light sources 152 may be configured to provide structured light, while another one or more of the light sources 152 may be configured to provide non-structured light.

Also, in some embodiments, the light source 152 may be integrated with one or more cameras 154. For example, in one implementation, the optical system 150 may include multiple pods, wherein each pod may have one or more light sources 152 and one or more cameras 154 (e.g., two cameras 154).

As shown in FIG. 1, the optical system 150 also includes a plurality of time-of-flight (TOF) cameras 158. Each TOF camera 158 is configured to provide depth image(s). A depth image has pixel values representing a distance between a reference point and a surface point detected. In some embodiments, each TOF camera 158 may be an infrared camera. During use, images from the cameras 154 and the TOF cameras 158 are processed by the processing unit 156 to obtain and monitor surface contours of the patient before and during treatment for the purpose of patient setup (absolute positioning and/or relative positioning), patient monitoring during treatment (e.g., monitoring absolute position and/or relative position), tool surveillance, prevention of patient-machine collisions, or a combination of the foregoing. Patient monitoring may include: (1) ensuring that the patient does not leave its setup position, and/or (2) recording a periodic patient motion due to breathing, and controlling a machine accordingly (e.g., beam hold, multi-leave collimator tracking, tracking of patient support, etc.).

In some cases, the TOF cameras 158 may help increase a field of view, and may observe blind spots not captured by the camera(s) 154.

In some embodiments, the TOF cameras 158 may provide images at lower resolution than that of the images provided by the cameras 154. In other embodiments, the TOF cameras 158 may provide images at higher resolution than that of the images provided by the cameras 154.

In the illustrated example, the optical system 150 has two TOF cameras 158. In other embodiments, the optical system 150 may include more than two (e.g., three, four, etc.) TOF cameras 158, or fewer than two (i.e., one) TOF camera 158.

The TOF cameras 158 may be moveably mounted to the frame 160. In one implementation, each of the TOF cameras 158 may be rotatably mounted to the frame 160 (e.g., via a ball joint) so that the TOF camera 158 is rotatable about one or more axes with respect to the frame 160. In other embodiments, instead of ball joints, the TOF cameras 158 may be moveably mounted to the frame 160 using other connectors, such as arms, so that the TOF cameras 158 are moveable with respect to the frame 160. In other embodiments, the one or more of the TOF cameras 158 may be mounted directly to the treatment system 10 or a room.

In other embodiments, the TOF cameras 158 may be mounted to different frame than that of the optical system 150. Also, in further embodiments, the TOF cameras 158 may be configured to be mounted to the medical system 10, e.g., to the gantry, to the patient support. In some cases, the TOF cameras 158 may be mounted to deployable arms that are coupled to the medical system 10. In other embodiments, the TOF cameras 158 may be mounted to a room (e.g., to a wall, a ceiling, a floor, etc.).

In other embodiments, the optical system 150 may not include any TOF cameras 158. In further embodiments, the optical system 150 may include TOF camera(s) 158, but not the cameras 154.

In some embodiments, the optical system 150 may include multiple pods, wherein each pod may have one or more light sources 152, one or more cameras 154 (e.g., two cameras 154), and one or more TOF cameras 158. For example, there may be a first pod having one or more light sources 152 and two cameras 154, and a second pod having one or more light source 152 and two cameras 154. In addition, in some embodiments, a pod may include another type or auxiliary camera or depth measurement device. For example, apart from TOF camera, a pod may include ultrasonic distance sensor(s), light sensitive guard(s), or laser scanner(s). In some embodiments, a pod may also include one or more regular video camera(s). In such cases, a processor may obtain information from the regular video camera(s), and merge that information with 3D images. The video cameras may be used to detect markers with known geometric properties to obtain additional geometric 3D information. In further embodiments, the optical system 150 may include a web camera in each pod. In some cases, the image from the web camera or regular video camera may be overlaid on a detected surface or distance map. This may help to define a region of interest. For example, if a user does not see a surface representation of a user interface screen, but can see a realistic photograph of the scene, then the user may still define the region of interest using the user interface.

In some embodiments, the pod(s) may be mounted to a frame of the optical system 150. In other embodiments, the pod(s) may be mounted to a different frame than that of the optical system 150. Also, in further embodiments, the pod(s) may be configured to be mounted to the medical system 10, e.g., to the gantry, to the patient support. In some cases, the pod(s) may be mounted to deployable arms that are coupled to the medical system 10. In other embodiments, the pod(s) may be mounted to a room (e.g., to a wall, a ceiling, a floor, etc.).

The optical system 150 may be configured to provide patient setup, patient monitoring, device monitoring, respiratory motion control, patient-machine collision prevention, or any combination of the foregoing. Thus, in some cases, the same optical system 150 may provide multiple purposes. In some embodiments, different clinical use cases mentioned above may be performed simultaneously. In one implementation, the sequence of real-time input images from the camera(s) 154 and from the TOF camera(s) 158 may be processed by the processing unit 156 to perform patient-machine collision prevention. The same real-time input images (or a subset of them) from the camera(s) 154, and the same real-time input images (or a subset of them) from the TOF camera(s) 158 may also be processed by the processing unit 156 to perform patient monitoring and/or device monitoring. Also, in some embodiments, by combining external surface information of the patient (provided by the optical system 150) with x-ray imaging of the internal anatomy, highly integrated and automated treatment workflows may be achieved.

Patient Setup

In one method of use, the light source 152 of the optical system 150 may be used to provide structured light. The structured light may be projected onto an object, such as a patient, for patient setup. As used in this specification, when light is described as being projected onto a patient, it is intended to cover the scenario in which the light is projected directly onto the patient (i.e., onto the skin of the patient), as well as the scenario in which the light is projected onto an object worn or coupled to the patient (e.g., onto a garment worn by the patient, a blanket covering the patient, a sticker on the patient, etc.). The cameras 154 sense the structured light as projected on the patient, and generate images of the projected structured light. The processing unit 156 is configured to process the images from the cameras 154, and determine a position (e.g., location and/or orientation) of the patient based on the processed images. Once the position of the patient is determined, the processing unit 156 may determine which direction to move the patient, and how much to move the patient, based on a desired position of the patient to be achieved.

In some cases, a reference image may be obtained by the processing unit 156. The reference image may be generated using the light source 152 and the cameras 154 during a treatment planning session, or on the day of treatment before the treatment session. The reference image includes an image of structured light as projected onto the patient, which indicates a desired position of the patient relative to some coordinate to be achieved. During the patient setup, the light source 152 and the cameras 154 are used to generate an input image. The processing unit 156 compares the input image with the reference image to determine if they match. If not, the patient is then positioned until the input image and the reference image match.

In some embodiments, if the optical system 150 includes one or more TOF cameras (e.g., the TOF cameras 158), the TOF camera(s) may generate one or more depth images. In such cases, the processing unit 156 may use the depth image(s) to perform patient setup. The processing unit 156 may use only the depth image(s) without the optical image(s) from the camera(s) 154. Alternatively, the processing unit 156 may use both depth image(s) and image(s) from the camera(s) 154 to perform patient setup. In one implementation, a reference depth image may be obtained by the processing unit 156. The reference depth image contains information regarding a desired position of a surface of a patient with respect to one or more objects (e.g., a component of the treatment system 10, the patient support 14, a wall of the room, etc.) surrounding the patient. The reference depth image may be generated by the TOF camera(s) during a treatment planning session, or on the day of the treatment before the treatment session begins. During a patient setup procedure, the TOF camera(s) provides depth image, which indicates a position of the surface of the patient with respect to one or more objects surrounding the patient. The processing unit 156 compares the depth image with the reference depth image to see if they match. If not, then the patient is positioned until the depth image matches the reference depth image.

Patient Position Monitoring

After the patient is setup, the treatment system 10 may then initiate treatment of the patient by delivering treatment energy towards the patient. During treatment, the light source 152 may continue to provide structured light onto the patient, and the cameras 154 may continue to sense the projected structured light to monitor the position of the patient. The processing unit 156 may compare the determined position of the patient with a reference position that is determined during the patient setup. If the position of the patient deviates from the reference position by more than a threshold, then the processing unit 156 may generate a control signal to stop the delivery of the treatment energy, to move the patient to a correct position, and/or to move the treatment beam accordingly.

In some cases, the act of monitoring the position of the patient may be performed without the processing unit 156 determining the actual coordinates of the patient. In one implementation, during treatment, the cameras 154 repeatedly generate input images indicating the structured light as projected onto the patient. The processing unit 156 processes these real-time input images by comparing each of them with the reference image. If the real-time input image matches with the reference image, then the processing unit 156 may determine that the patient has not moved, and the treatment may be allowed to continue. On the other hand, if the real-time image does not match the reference image, then the processing unit 156 may determine that the patient has moved, and the delivery of treatment energy may be stopped (e.g., in response to the processing unit 156 generating a control signal to stop the delivery of treatment). Alternatively, instead of stopping the delivery of treatment energy, the processing unit 156 may generate one or more control signals to move the patient support (couch tracking) and/or to move the beam, in order to compensate for the amount of patient movement. Movement of the beam may be accomplished by beam steering and/or by operation of the collimator.

In some embodiments, the comparing of the input image with the reference image may be performed by the processing unit 156, which performs pattern matching. In some cases, the comparison may be achieved by the processing unit 156 performing cross-correlation between the input image and the reference image. The cross-correlation results in a correlation value, which indicates a degree of match between the two-dimensional input image and the two-dimensional reference image. If the correlation value exceeds a certain pre-determined threshold (e.g., 0.9), then the processing unit 156 may determine that there is no patient movement. On the other hand if the correlation value is below a certain threshold, then the processing unit 156 may determine that the patient has moved. In other embodiments, an iterative closest point (ICP) algorithm may be used when processing the input image and the reference image.

In other embodiments, the light source 152 provides structured light and directs it onto an object, and the reflected light (e.g., IR light) from the object is measured by two cameras 154 which are offset from the light source 152. The geometry of the light source 152 and the two offset cameras 154 is known. Accordingly, the processing unit 156 can use triangulation to calculate the distance of surface by finding the same structured pattern in the images from both cameras 154. The result is a depth map (or distance map), similar to the TOF technology. In some cases, the light source 152 and the two cameras 154 may be implemented as one pod, and there may be additional pod(s), wherein each pod has a light source and two offset cameras. The processing unit 156 may be configured to add the depth map from one pod to other depth map(s) determined from other pod(s) at other locations in order to map out the surface of the object, thereby forming a larger depth map. In some cases, this depth map may be represented by a point cloud in a defined coordinate system. The processing unit 156 may also calculate the distance of a reference surface to a measured surface to detect a possible offset.

In some embodiments, the structured pattern may be implemented using time-varying gray levels. In such cases, the time-varying gray levels are projected by a light source on the surface to be measured. The processing unit 156 then utilizes an algorithm to find the corresponding pixel in both camera images. Knowing the camera pixel for this surface point and the cameras configuration (e.g., position and/or orientation of each camera in the pod), the angle of the ray towards this object point can be determined by the processing unit 156 for each camera. As the distance between both cameras in the pod is known, triangulation technique may then be used by the processing unit 156 to calculate the distance to this surface point (also known as “distance of surface”). In some embodiments, such distance to the surface point may be measured from the camera pod. The above process may be repeated for all object points to thereby create a depth/distance map, which represents a surface of interest in a known coordinate system.

In one implementation, each of the cameras in a given pod records a series of images with different fringe patterns projected onto the patient/object of interest. From those images, a disparity map is then created by the processing unit 156. A disparity map measures the distance of two corresponding points as seen by the two cameras. These disparity maps are then used by the processing unit 156 to create a 3D ordered point cloud, i.e. a surface information of the object that is seen by both cameras (in a given coordinate system). With multiple pods, such 3D ordered point clouds may be merged to a bigger common surface by the processing unit 156. The bigger common surface is advantageous because it fills gaps of areas that are not seen by one or several pods, and it can increase the overall field of view.

In some embodiments, in addition or in the alternative to using input image(s) from the camera(s) 154, if the optical system 150 includes TOF camera(s), the processing unit 156 may use depth images from the TOF camera(s) for patient monitoring. For example, during treatment, the TOF camera(s) repeatedly generate input depth images indicating the surface profile of the patient. The processing unit 156 processes these real-time depth images by comparing each of them with a reference depth image. If the real-time depth image matches with the reference depth image, then the processing unit 156 may determine that the patient has not moved, and the treatment may be allowed to continue. On the other hand, if the real-time depth image does not match the reference depth image, then the processing unit 156 may determine that the patient has moved, and the delivery of treatment energy may be stopped (e.g., in response to the processing unit 156 generating a control signal to stop the delivery of treatment).

Respiratory Phase Determination and Treatment Control

Also, during treatment, the light source 152 may project the structured light onto a moving part (e.g., torso) of the patient for respiratory phase and amplitude determination and treatment control. In such cases, the cameras 154 sense the structured light as projected onto the torso of the patient, and the processing unit 156 then determines a respiratory phase and amplitude based on the images from the cameras 154.

In one implementation, the light source 152 provides structured light and directs it onto an object, and the reflected light (e.g., IR light) from the object is measured by two cameras 154 which are offset from the light source 152. The geometry of the light source 152 and the two offset cameras 154 is known. Accordingly, the processing unit 156 can use triangulation to calculate the distance of surface based on the structured pattern as they appear in the images from both cameras 154. The result is a depth map. In some cases, the light source 152 and the two cameras 154 may be implemented as one pod, and there may be additional pod(s), wherein each pod has a light source and two offset cameras. The processing unit 156 may be configured to add the depth map from one pod to other depth map(s) determined from other pod(s) at other locations in order to map out the surface of the object, thereby forming a larger depth map. In some cases, the depth map (whether determined using one pod or multiple pods) may be represented by a point cloud in a defined coordinate system. The mapped out surface (as represented by the point cloud) may be used by the processing unit 156 to determine amplitude or a change in amplitude (e.g., due to breathing). The processing unit 156 may be configured to repeat the above process as real-time images from the cameras 154 are received by the processing unit 156. This results in a determination of breathing amplitudes over time. In some embodiments, the processing unit 156 may use the determined amplitudes to determine breathing phases of the patient.

In another implementation, before a treatment session, images of the structured light as projected onto the patient may be generated and recorded as reference images. The reference images form a video showing how the structured light pattern changes during a breathing cycle of the patient. During treatment, the cameras 154 provide real-time input images of the structured light as projected onto the patient while the patient is breathing. The processing unit 156 may process each real-time input image by finding one of the reference images that matches with the real-time input image. The respiratory phase for the real-time input image may then be determined as the same respiratory phase as the matched reference image. For example, if the matched reference image was generated when the patient is at breathing phase=3.6 (or during phase range from 3.0-4.0), then the real-time input image may be considered as being generated when the patient is at the same breathing phase or phase range.

In some embodiments, the determined respiratory phase or amplitude may be used to control the treatment system 10. For example, the determined respiratory phase or amplitude may be used to gate a delivery of a treatment beam. In one implementation, if the determined respiratory phase or amplitude is within a prescribed range of phases or amplitudes for delivering treatment, then the treatment system 10 is operated by the processing unit 156 (which provides a control signal) to deliver the treatment beam. On the other hand, if the determined respiratory phase or amplitude is outside the prescribed range of phases or amplitudes for delivering treatment, then the treatment system 10 is operated by the processing unit 156 (which provides a control signal) to stop the delivery of treatment beam.

In other embodiments, the determined respiratory phase or amplitude may be used to control the treatment system 10 so that the delivery of the treatment beam is in synchronization with a respiratory movement of the patient. For example, the processing unit 156 may generate one or more signal to control a delivery of the treatment beam so that the treatment beam follows the movement of the patient.

In some embodiments, in addition or in the alternative to using input image(s) from the camera(s) 154, if the optical system 150 includes TOF camera(s), the processing unit 156 may use depth images from the TOF camera(s) to determine breathing phase or amplitude and to control the treatment system 10 based on the determined breathing phase. For example, during treatment, the TOF camera(s) repeatedly generate input depth images indicating the surface profile of the patient. The processing unit 156 processes these real-time depth images to determine a breathing amplitude associated with each real-time depth image. In one implementation, as the patient breaths, the torso will move up and down. The real-time depth images will capture the changing positions of the torso while the patient breaths. Based on the breathing amplitude, the processing unit 156 may then determine a corresponding breathing phase. The processing unit 156 may also use the breathing phase to control the treatment system 10, as similarly discussed.

Respiratory Motion Control

In some embodiments, the optical system 150 may be configured to provide respiratory motion control. In some embodiments, the light source 152 may be configured to project a structured light onto the patient. The cameras 154 generate image(s) of the structured lights as projected onto the patient, and provide such images to the processing unit 156 for processing. The processing unit 156 analyzes the image(s) to determine whether a certain respiratory phase/amplitude has been achieved by the patient. For example, the processing unit 156 may determine whether a certain breath-hold amplitude has been achieved based on an analysis of the image. If a desired breath-hold amplitude has been achieved, then processing unit 156 may generate a signal to allow a medical procedure to be performed. For example, the processing unit 156 may generate a signal to allow a treatment beam to be delivered by the treatment system 10. In some embodiments, the processing unit 156 may analyze a number of images from the cameras 154 overtime, to determine whether a certain breath-hold amplitude has been achieved for a certain duration (e.g., 2 seconds, 3 seconds, etc.). If a desired breath-hold amplitude has been achieved for a certain prescribed duration, then processing unit 156 may generate a signal to allow a medical procedure to be performed. For example, the processing unit 156 may generate a signal to allow a treatment beam to be delivered by the treatment system 10.

In some embodiments, in addition or in the alternative to using input image(s) from the camera(s) 154, if the optical system 150 includes TOF camera(s), the processing unit 156 may use depth images from the TOF camera(s) to perform respiratory motion control. For example, the TOF camera(s) may generate input depth image indicating the surface profile of the patient. The processing unit 156 processes the real-time depth image to determine whether a certain respiratory control has been achieved by the patient. If a desired breath-hold amplitude has been achieved, then processing unit 156 may generate a signal to allow a medical procedure to be performed. For example, the processing unit 156 may generate a signal to allow a treatment beam to be delivered by the treatment system 10. In some embodiments, the processing unit 156 may analyze a number of depth images from the TOF camera(s) overtime, to determine whether a certain breath-hold amplitude has been achieved for a certain duration (e.g., 2 seconds, 3 seconds, etc.). If a desired breath-hold amplitude has been achieved for a certain prescribed duration, then processing unit 156 may generate a signal to allow a medical procedure to be performed. For example, the processing unit 156 may generate a signal to allow a treatment beam to be delivered by the treatment system 10.

Device Monitoring

It should be noted that the techniques described above should not be limited to monitoring the patient, and that they can also be employed to monitor device(s). In some embodiments, the light source 152 may be configured to project structured light onto one or more device surface(s). The device surface(s) may be a surface of the patient support 14, a surface of the housing of the treatment system 10, a surface of a gantry, a surface of the energy source, a surface of an imaging device, a surface of a positioning device, a surface of an accessory (e.g., a positioning device, such as the Calypso, or any combination of the foregoing. In some cases, the accessory may be a positioning device, such as the Calypso device 180 (available at Varian, Palo Alto, Calif.) shown in FIG. 1. Also, in some embodiments, the accessory may be considered to be a part of the system 10.

During treatment, the light source 152 may continue to provide structured light onto the device(s), and the cameras 154 may continue to sense the projected structured light to monitor the position(s) of the device(s). The processing unit 156 may compare the determined position(s) of the device(s) with respected desired position(s). If the position(s) of the device(s) deviates from the desired position(s) by more than a threshold, then the processing unit 156 may generate a control signal to stop the delivery of the treatment energy.

In some embodiments, in addition or in the alternative to using input image(s) from the camera(s) 154, if the optical system 150 includes TOF camera(s), the processing unit 156 may use depth images from the TOF camera(s) for device(s) monitoring. For example, during treatment, the TOF camera(s) repeatedly generate input depth images indicating the surface profile(s) of the device(s). From the determined surface profile(s), the processing unit 156 may determine the position(s) of the device(s). If the position(s) of the device(s) deviates from the desired position(s) by more than a threshold, then the processing unit 156 may generate a control signal to stop the delivery of the treatment energy.

Patient-Machine Collision Prevention

In some embodiments, the optical system 150 may be configured to provide patient-machine collision prevention. In some embodiments, the light source 152 may be configured to project a first structured light onto the patient, and a second structured light onto one or more device surface(s). The device surface(s) may be a surface of the patient support 14, a surface of the housing of the treatment system 10, a surface of a gantry, a surface of the energy source, a surface of an imaging device, a surface of a positioning device, a surface of an accessory, or any combination of the foregoing. The cameras 154 generate images of the structured lights as projected onto the patient and onto the device(s), and provide such images to the processing unit 156 for processing. The processing unit 156 analyzes the images to determine the position of the patient and the position(s) of the device(s) being monitored. If the positon of the patient and the position of one of the device is too close (e.g., is less than a prescribed threshold), then the processing unit 156 may determine that there may be a risk of collision, and may generate a control signal to stop the operation of the treatment system 10. The processing unit 156 may also generate an indicator for informing a user that there is a risk of collision. The indicator may be in a form of a visual indicator (e.g., a light, a display of an object in a screen, etc.), an audio indicator (e.g., an alarm), or both.

In some embodiments, in addition or in the alternative to using input image(s) from the camera(s) 154, if the optical system 150 includes TOF camera(s), the processing unit 156 may use depth images from the TOF camera(s) to perform patient-machine collision prevention. For example, during treatment, the TOF camera(s) repeatedly generate input depth images indicating the surface profile of the patient. The processing unit 156 processes these real-time depth images to determine if there is another object that has been moved too close (e.g., less than a prescribed threshold) to the surface of the patient. If so, then the processing unit 156 may determine that there may be a risk of collision, and may generate a control signal to stop the operation of the treatment system 10. The processing unit 156 may also generate an indicator for informing a user that there is a risk of collision. The indicator may be in a form of a visual indicator (e.g., a light, a display of an object in a screen, etc.), an audio indicator (e.g., an alarm), or both. In one implementation, the processing unit 156 may be configured to monitor a layer of space that is next to the surface of the patient. For example, the layer of space may be the space within 12 (or less) inches from the surface of the patient. If there is no object within the layer of space, the input depth image will have corresponding pixel values indicating such. On the other hand, if an object has been moved into the layer of space, the input depth image will sense the surface of the object in the layer of space, indicating that the object has been moved too close to the patient.

In the above embodiments, the optical system 150 has been described with reference to providing structured light. In other embodiments, the light source 152 of the optical system 150 may be configured to provide non-structured light. The non-structured light may be projected onto the patient, onto a marker device coupled to the patient, onto a component of the treatment system 10, onto a marker device coupled to the treatment system 10, or any combination of the foregoing.

It should be noted that the optical system 150 is advantageous because it uses non-visible light for determining position(s) of patient and/or position(s) of machine components.

In other embodiments, instead of providing infrared light, the optical system 150 (e.g., the light source 152) may be configured to provide ultraviolet light. In further embodiments, the optical system 150 may be configured to provide other light(s) that is non-visible. For example, in other embodiments, the light source 152 may be configured to provide non-visible light having wavelength(s) that is outside the infrared range, and/or the ultraviolet range.

Also, providing a single optical system 150 for patient setup, patient monitoring, tool surveillance, and patient-machine collision is advantageous because it obviates the need to use separate different systems for these purposes. The optical system 150 is also advantageous because it obviates the need to have a separate system for detecting respiration characteristics (e.g., phases, amplitudes, period, etc.).

In some embodiments, the system 10 may be configured to provide kV and/or MV imaging. For example, the energy source 20, or another energy source may be configured to provide energy at kV range (e.g., having an energy level anywhere from 1-999 kV) or at MV range (e.g., having an energy level anywhere from 1 MV to 20 MV) for imaging purpose. In such cases, the optical system 150 in combination with the kV/MV imaging capability may provide an unprecendented close control and correlation between internal object (e.g., a target or a critical organ inside the patient) and external fiducials (e.g., external markers, such as any of the markers described herein). For example, the processing unit 156 of the optical system 150 may process images from the cameras to determine a surface. The surface may correspond with an abdominal region of the patient, and/or a chest of the patient. In such cases, the processing unit 156 may correlate a position of the determined surface to an internal region of the patient. The internal region of the patient may be determined or represented using images of internal parts of the patient, such as x-ray, CT, CBCT, etc.

Patient Identification

In some embodiments, the optical system 150 may also be configured to provide patient identification. For example, one or more images obtained by the cameras 154 may be transmitted to processing unit 156, which processes the image(s) to determine an identity of the patient. For example, the processing unit 156 may perform pattern matching to determine whether features in an image match those in a reference image (which may be a previously obtained image of the patient). If the features match, then the processing unit 156 may determine that the identity of the patient is confirmed, and may allow additional procedures (e.g., patient setup, imaging, treatment, etc.) to be performed.

Other Use Cases

It should be noted that the optical system 150 may have other use cases in other embodiments. For example, in other embodiments, the optical system 150 may be used for treatment planning. In some cases, the optical system 150 may be used in a CT simulation procedure in order to determine a patient surface, which is then used for treatment planning. During the CT simulation process, the patient surface is determined using the optical system 150 while the patient is in a treatment position using positioning aids (such as breast boards, etc.). The light source 152 of the optical system 150 provides structured light, while two cameras 154 are used to detect the reflected structured light. The images of the reflected structured light are then processed by the processing unit 156 to determine the patient surface. The processing unit 156 may also use the patient surface for treatment planning. For example, the processing unit 156 may select beam incidences which will not lead to a collision between the patient and other components of a treatment machine.

In other embodiments, the optical system 150 may be used in a treatment room that includes a CT machine moveably supported on rails. In such cases, the optical system 150 may be configured to provide all of the features described herein. For example, the light source 152 may be configured to provide structured light and/or non-structured light, and the cameras 154 are used to detect the reflected light. Images from the cameras 154 are then processed by the processing unit 156 to perform various features described with reference to the different use cases in the above embodiments. Also, in some embodiments, the patient on the patient support may be moved to an imaging position associated with the CT machine, and may be imaged by the CT machine in the treatment room. Such may be accomplished by moving the patient support and/or the CT machine along the rails. After the patient is imaged, the patient may then be moved (e.g., by moving the patient support) to a treatment position associated with the treatment machine in the treatment room. The optical system 150 may be configured to monitor the patient position while the patient is moved between the imaging position and the treatment position. If the patient has moved relative to the patient support, the optical system 150 may generate a signal to inform an operator. In response, the operator may re-image the patient before treatment is initiated. Also, the optical system 150 may be used to detect patient movement that may occur while the patient is being switched between the imaging position and the treatment position. The optical system 150 may also be used to monitor the patient after the patient is moved to a treatment position while being supported on the patient support.

In other embodiments, instead of, or in addition to, CT machine, the treatment room may include other types of imaging devices. By means of non-limiting examples, the treatment room may include a C-arm imaging system, a PET/CT machine, a MRI machine, or any other imaging device. The imaging device may be stationary (such that it is fixed to the floor or ceiling), or may be moveable relative to the floor.

Also, in other embodiments, the optical system 150 may be used in a room that includes any imaging device. For example, the imaging device may be a CT machine in a treatment room or imaging room. The CT machine may be stationary, or may be moveable on rails. In such cases, the optical system 150 may be configured to provide all of the features (e.g., patient monitoring, collision avoidance, etc.) described herein. In some embodiments, in response to input by the camera system, the processing unit 156 of the optical system 150 may trigger an imaging system to obtain one or more image(s), such as re-CT image(s), re-sim image(s), CT-sim image(s), etc. The optical system 150 may also be used to monitor the patient during operation of the imaging system to obtain any of the above images.

Re-CT image refers to a CT image that is re-done or repeated, such as for treatment. This term helps differentiate the additional, repetitive acquired CT patient data set taken while the patient is already under treatment, from the first planning or simulation CT (which is used for treatment planning). The anatomy of the patient during treatment may change (e.g., due to weight loss, tumor changes, etc.), which changes the dose distribution significantly. Therefore a new treatment plan may be needed, and the so-called re-CT is acquired for such purpose.

Re-sim image refer to a simulation image for treatment planning that is repeated while the patient is under treatment. The simulation image may be a CT image, a x-ray/fluoro image, or any of other types of image. As mentioned, sometimes it is needed to acquire new image while the patient is under treatment to accommodate for anatomical changes. Re-sim image may be used for such purpose.

CT sim image refers to image used for treatment simulation performed during treatment planning. In some cases, for CT sim imaging, the optical system 150 may be used to detect the patient surface while the patient is in treatment position using positioning aids (such as breast boards, etc.). The detected patient surface may then be used for the planning process in order to select beam incidences which will not lead to a collision later.

In further embodiments, the optical system 150 may be used to continuously determine the positions of the patient surface in real time as the patient is undergoing periodic physiological movement (e.g., breathing movement). For example, the camera(s) of the optical system 150 may detect the patient surface at the body as the surface is moving up and down due to breathing motion. The processing unit 156 may process the position of the patient surface to determine breathing amplitudes and/or breathing phases. In some cases, such may be performed in real time so that the determined breathing characteristics may be used by the processing unit 156 to control a treatment machine. For example, the processing unit 156 may generate one or more control signals to control the treatment machine so that delivery of treatment energy corresponds with the breathing characteristics. In one implementation, the treatment machine may be a radiation treatment machine, and the processing unit 156 may control a gantry, an energy source, a patient support, a collimator, or any combination of the foregoing, based on processing of the images from the optical system 150. The processing unit 156 may gate a delivery of the treatment energy based on the processing of the images.

Marker Apparatus

In some embodiments, the optical system 150 may also include one or more markers for viewing by the cameras 154. During use, the light source 152 of the optical system 150 provides non-structured light and projects the non-structured light towards the marker(s). The non-structured light may have a wavelength that is in the infrared range. In some cases, the non-structured light does not have any wavelength that is outside the infrared range. In other cases, the non-structured light may have wavelengths that are outside the infrared range. The marker(s) reflects the non-structured light (reflected off from the markers), and the cameras 154 detect the reflected non-structured light.

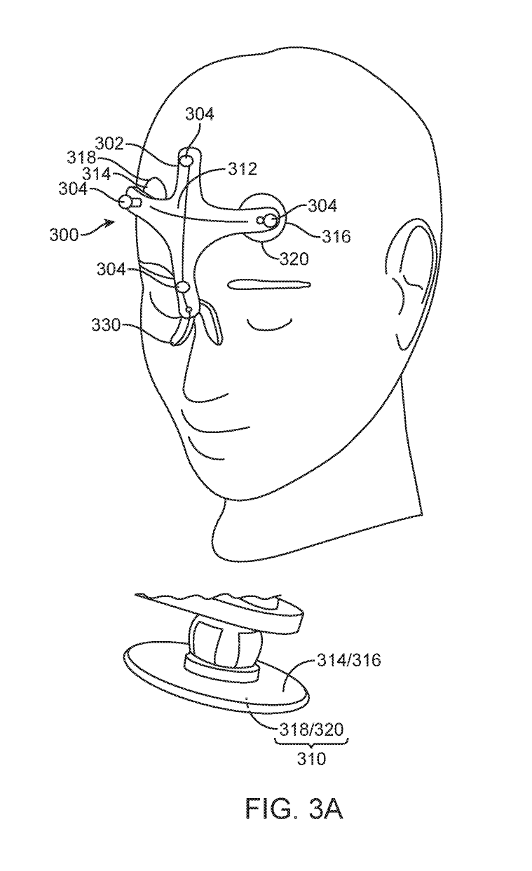

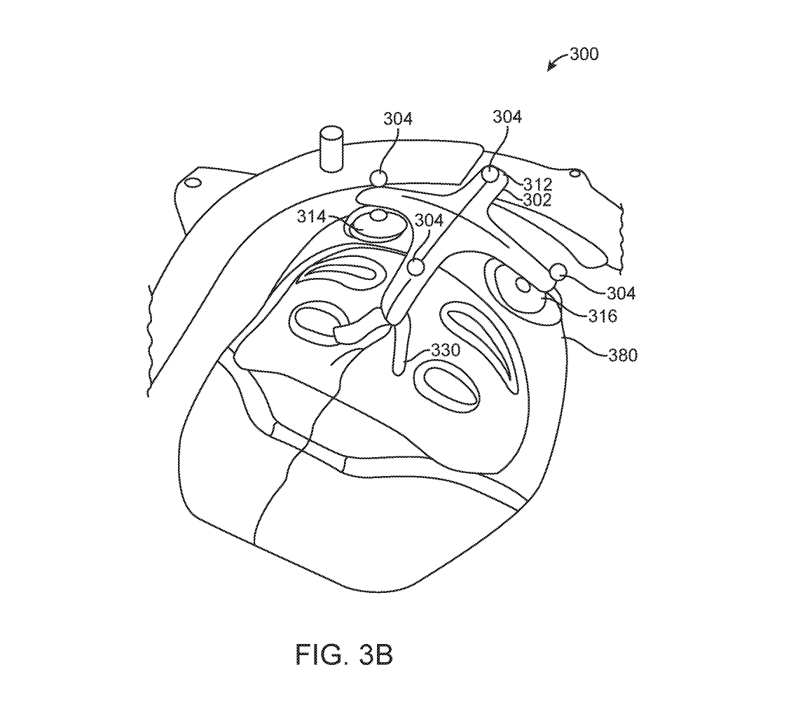

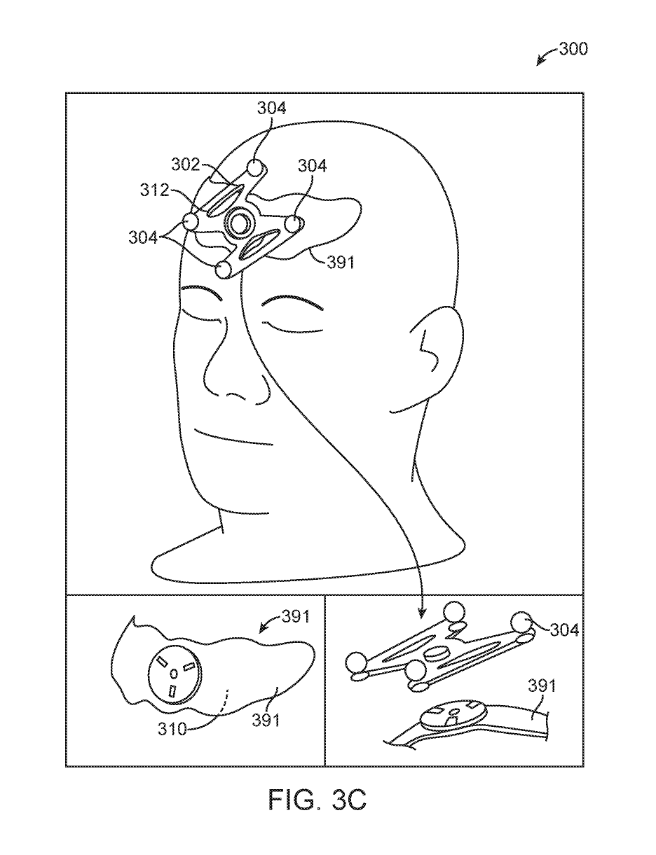

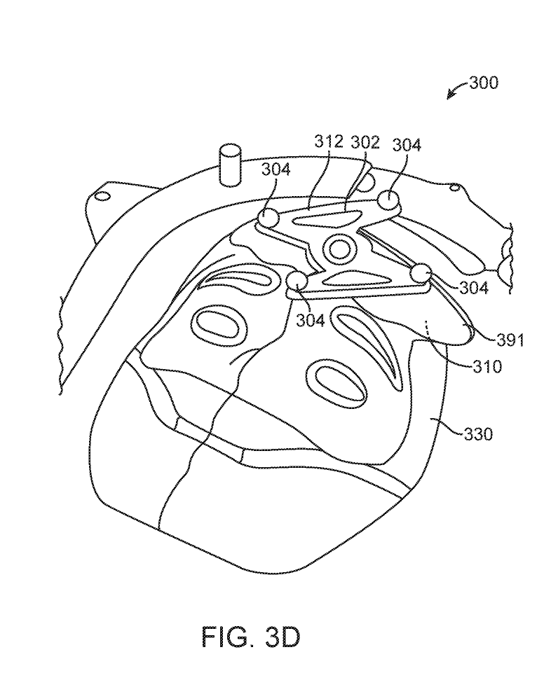

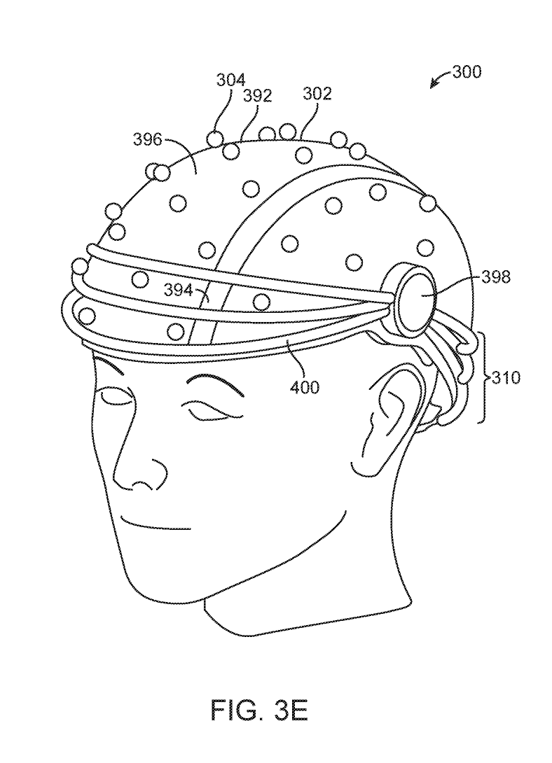

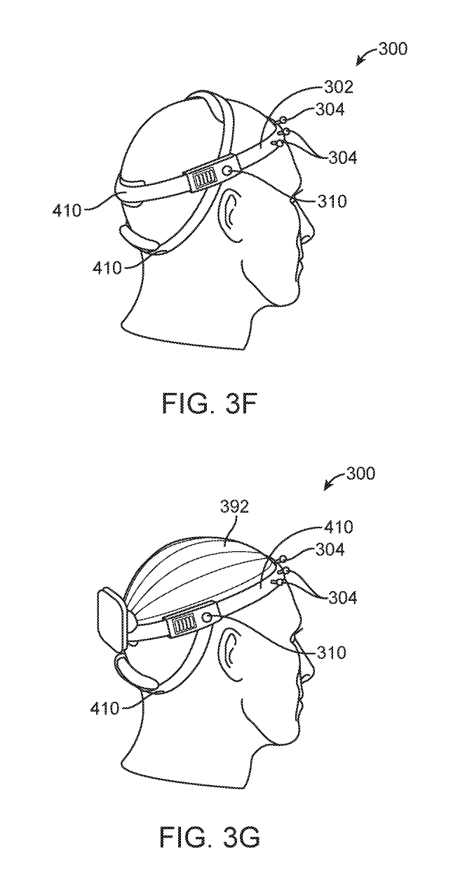

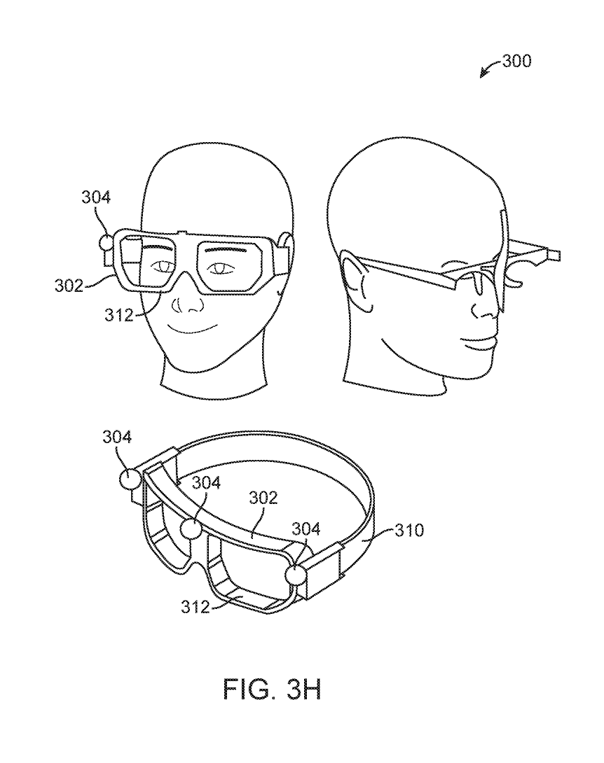

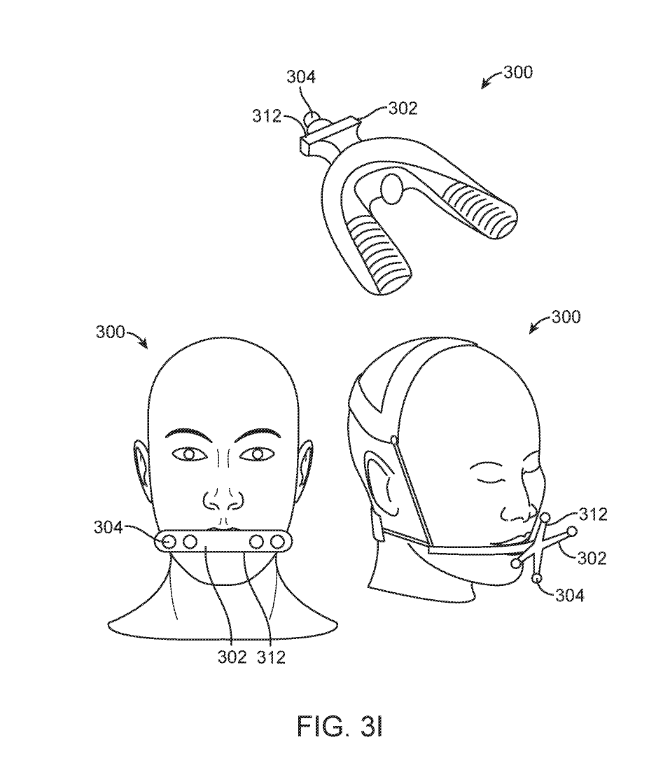

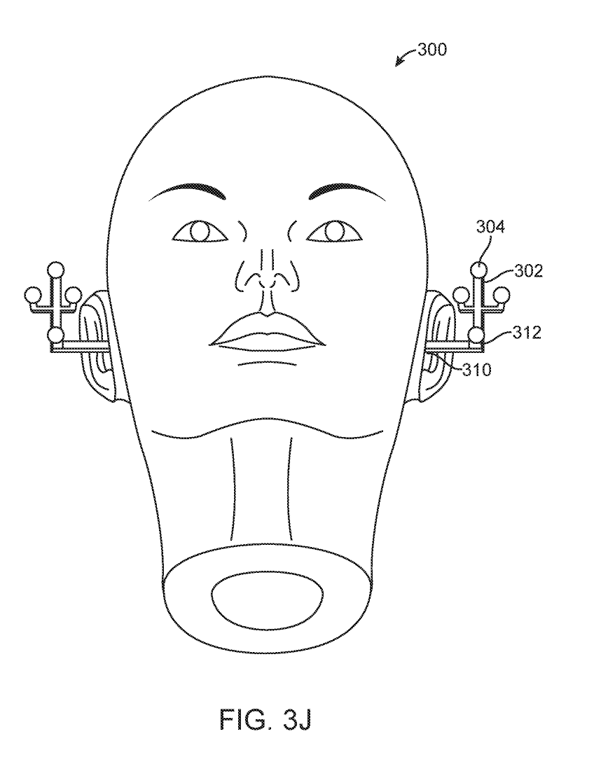

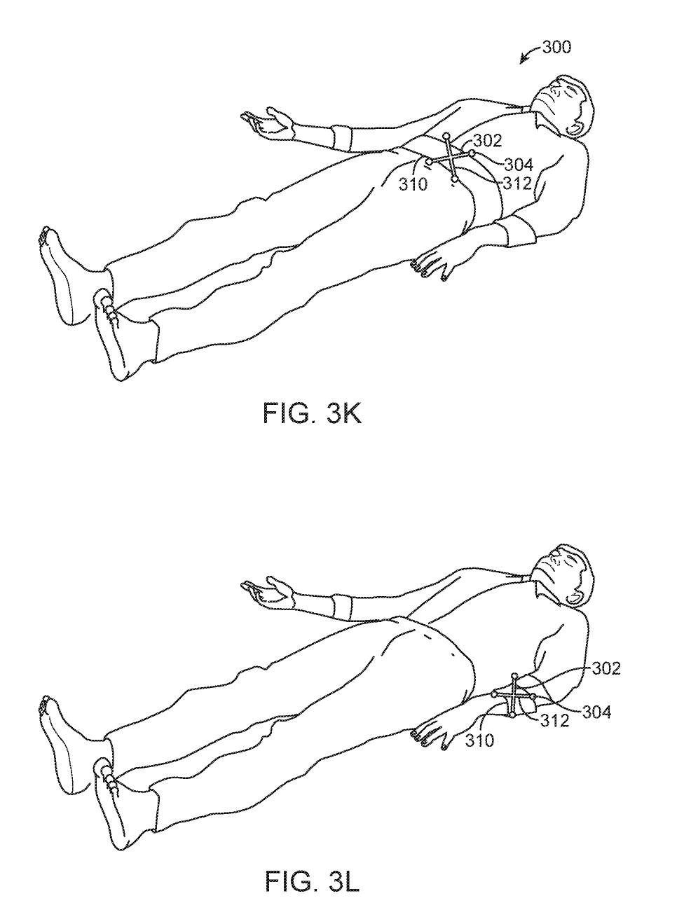

In some embodiments, the marker(s) may be implemented as a patient-mounted device. FIGS. 3A-3F illustrate examples of patient-mounted devices that include multiple markers. During use, the light source 152 projects light onto the markers, and the cameras 154 sense the markers and generate corresponding image(s) of the markers. In some cases, the light provided by the light source 152 may be non-structured light in the infrared range (e.g., the light has wavelengths that are only in the infrared range). In such cases, the cameras 154 are configured to capture light in the infrared range that is reflected from the markers. In other embodiments, the light source 152 may provide light in other wavelengths that are different from the infrared wavelengths, and the cameras 154 may be configured to capture light having other wavelengths.

FIG. 3A illustrates an apparatus 300 having a structure 302 for coupling to a patient, and multiple infrared reflective markers 304 coupled to the structure 302, wherein the infrared reflective markers 304 have fixed positions relative to each other. In other embodiments, the infrared reflective markers 304 may not have fixed positions, and one or more markers 304 may move relative to one or more other markers 304. The apparatus 300 also includes a securing mechanism 310 configured to secure the structure 302 relative to the patient. In the illustrated example, the structure 302 includes a frame 312, a first base 314, and a second base 316. The first base 314 includes a first adhesive 318 for attachment to the patient, and the second base 316 includes a second adhesive 320 for attachment to the patient. The first adhesive 318 and the second adhesive 320 may be considered examples of the securing mechanism 310 that secures the structure 302 relative to the patient.

The frame 312 is advantageous because it provides a rigid platform to rigidly couple the markers 304 in fixed positions relative to each other. The frame 312 is small, lightweight, and provides patient comfort. In some cases, the frame 312 may be made from low density material to provide a lightweight structure. Also, the apparatus 300 is advantageous because it provides minimal patient contact. In some embodiments, the entire apparatus 300 is disposable. In other embodiments, the first base 314 and the second base 316 are disposable, while the frame 312 may be re-used.

In some embodiments, the first base 314 may be detachably coupled to the frame 312, and the second base 316 may be detachably coupled to the frame 312. In other embodiments, the first and second bases 314, 316 may be fixedly coupled to the frame 312. Also, in the illustrated embodiments, the first base 314 is rotatably coupled to the frame 312 so that the first base 314 is rotatable about one or more axes with respect to the frame 312. Similarly, the second base 316 is rotatably coupled to the frame 312 so that the second base 316 is rotatable about one or more axes with respect to the frame 312. Such configuration provides swivel bases that accommodate a majority of patient head sizes and shapes. In other embodiments, the first base 314 and the second base 316 may be coupled to the frame 312 in fixed positions.

In the illustrated example, the apparatus 300 has four infrared reflective markers 304. In other embodiments, the apparatus 300 may include more than four infrared reflective markers 304, or fewer than four infrared reflective markers 304. Also, in some embodiments, the markers may be radiopaque markers.

Also, as shown in the figure, the apparatus 300 further includes a nose piece 330 coupled to the frame 312. The nose piece 330 may be moveably coupled to the frame 312 (e.g., via a ball joint or a flexible joint) so that the nose piece 330 is rotatable about one or more axes (e.g., at least two axes) with respect to the frame 312. Also, in some embodiments, the nose piece 330 may be detachably coupled to the frame 312. This allows the nose piece 330 to be exchanged with another nose piece having a different size and/or shape to fit a specific patient.

In addition, as shown in the figure, the structure 302 also includes one or more laser alignment mark(s). During use, after the apparatus 300 has been mounted to the patient, the patient may be positioned to align the laser alignment mark(s) with one or more corresponding laser(s). This allows the patient to be positioned to a desired positon for treatment.

During treatment, the markers 304 of the apparatus 300 function as surrogate for target motion. In particular, the light source 152 projects infrared non-structured light onto the infrared reflective markers 304. The infrared reflected light is then captured by the camera(s) 154. The camera(s) 154 provides input image(s) to the processing unit 156. The processing unit 156 processes the image(s) to determine the positions of the markers 304. Based on known relative positions among the markers 304, the processing unit 156 can then determine the position of the apparatus 300. The above technique may be repeated so that the processing unit 156 can repeatedly process real-time input images from the camera(s) 154 and determines the real-time positions of the surrogate. As used in this specification, the term “real-time position” refer to a position of an item that is determined a short time (e.g., 1 second or less, and more preferably, less than 0.5 second, and more preferably less than 0.3 second, and even more preferably less than 0.1 second) after an image of the item is obtained. During treatment, the processing unit 156 can provide the determined position as surrogate for the position of the target desired to be treated.

As shown in the above example, the apparatus 300 is advantageous in that it functions as a rigid body that provides a surrogate for the target being treated. The rigid body may be stable when attached to the patient. The rigid body may have a low profile and/or low mass for patient's acceptance. The rigid body may be decoupled or separate from known immobilization accessories. Alternatively, the rigid body may be coupled to, or integral with, known immobilization accessories. The rigid body may be easily decoupled from the patient, which provides for fast, accurate, and easy attachment anytime before, during, and/or after a medical procedure. The rigid body may also allow the treatment system 10 to provide high accuracy tracking (e.g., sub-mm resolution tracking). The rigid body may also allow patient setup and/or real-time tracking of the target to be performed with high accuracy (e.g., sub-mm accuracy).

It should be noted that the apparatus 300 is not limited to having the above configuration, and that the apparatus 300 may have other configurations in other embodiments.

In some embodiments, the apparatus 300 may further include a mask 380 (FIG. 3B). The mask 380 may be considered to be a part of the structure 302. In other cases, the mask 380 may be considered to be a part of the securing mechanism 310 for securing the structure 302 relative to the patient.

It should be noted that the apparatus 300 is not limited to the configurations described, and that the apparatus 300 may have other configurations in other embodiments. For example, in other embodiments, the structure 302 of the apparatus 300 may not have multiple bases. Instead, the structure 302 may include a single base 391 (FIG. 3C). The base 391 may be configured to detachably couple to the frame 312 (e.g., via a snap connector, such as a mono coupling lock). Use of mono coupling is advantageous in that it allows the frame 312 to be easily detached and attached to the base 391 in repeatable manner. In some cases, the base 391 may be coupled to, or may be a part of, a mask 330 (FIG. 3D).

In further embodiments, the structure 302 may be in a form of a hat 392 (FIG. 3E). As shown in the figure, the hat 392 may include a first portion 394 made from a first material, and a second portion 396 made from a second material, wherein the first material is more elastic than the second material. In one implementation, the first portion 394 may be a compliant and elastic mesh, and the second portion 396 may be a scaffold that is more rigid than the first portion 394. In the illustrated example, the securing mechanism 310 includes a tensioning mechanism 398 configured to apply tension to one or more straps 400 around the hat 392 to thereby secure the hat 392 relative to the patient.

In other embodiments, instead of the hat 392, the apparatus 300 may include other types of devices for coupling to a head of the patient. For example, as shown in FIG. 3F, in other embodiments, the structure 302 of the apparatus 300 may include one or more straps 410 for placement around the head of the patient. The strap(s) 410 may include a strap connector or a strap tightening knob for securing the strap(s) relative to the head of the patient. In such cases, the strap connector or the strap tightening knob may be considered examples of the securing mechanism 310.

In further embodiments, the structure 302 of the apparatus 300 may include both the hat 392 and the one of more straps 410 (FIG. 3G).

Also, in other embodiments, the structure 302 of the apparatus 300 may be in the form of an eyewear (e.g., glasses, goggle, etc.) (FIG. 3H). The eyewear may include a strap or temples for securing the structure 302 relative to the head of the patient.

In still further embodiments, the structure 302 of the apparatus 300 may include a mouthpiece (FIG. 3I).

In other embodiments, the structure 302 may be configured for coupling to a skin above an ear of the patient (FIG. 3J). In one implementation, the apparatus 300 may include an earplug for placement in an ear canal of the patient. The structure 302 may also include a base with an adhesive for attachment to the skin above the ear of the patient.

In should be noted that the apparatus 300 is not limited to being coupled to a head of a patient, and may be configured to couple to other body parts (e.g., a neck, a torso, a limb, etc.) of the patient in other embodiments. For example, as shown in FIG. 3K, the apparatus 300 of FIG. 3A may be coupled to the torso of the patient. Also, in some embodiments, for torso application, the apparatus 300 may be made larger so that it covers a larger area across a majority of the width of the torso. In some embodiments, the apparatus 300 may be attached to breast. In further embodiments, the apparatus 300 may be attached to any soft tissue.

FIG. 3L illustrates an apparatus 300 for coupling to a limb of the patient that includes multiple markers. The structure 302 of the apparatus 300 may be configured to detachably couple to the patient. As shown in the figure, the securing mechanism 310 is in a form of a strap for strapping around the limb of the patient.

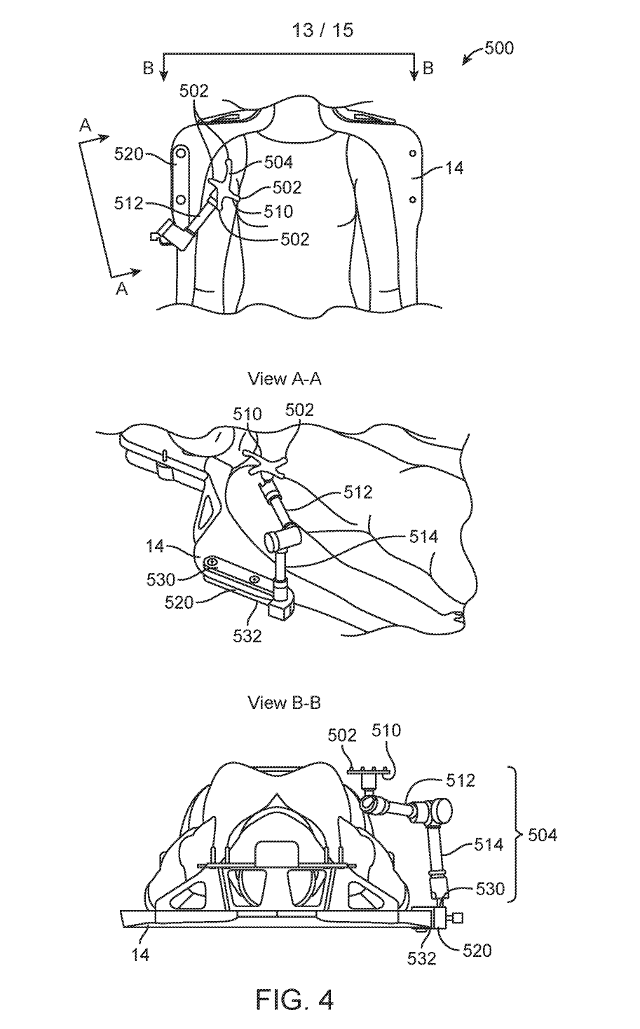

In some embodiments, a device-mounted marker apparatus may be provided. FIG. 4 illustrates an apparatus 500 that includes multiple markers 502. The apparatus 500 is configured to be mounted to the patient support 14. The apparatus 500 includes a structure 504 to which the markers 502 are secured. The structure 504 includes a frame 510, a first arm 512 moveably attached to the frame 510, and a second arm 514 moveably attached to the first arm 512. In the illustrated example, the connection between the frame 510 and the first arm 512 is a ball-joint, which allows the frame 510 to rotate relative to the first arm 512 around multiple axes. The connection between the first arm 512 and the second arm 514 comprises a single-axis rotation connector. In other embodiments, the connection between the first arm 512 and the second arm 514 may be other types of connection, such as a ball-joint.

The apparatus 500 also includes a securing mechanism 520 configured to detachably attach to the patient support 14. The second arm 514 is rotatably coupled to the securing mechanism 520 via a ball joint. In other embodiments, the second arm 514 may be moveably coupled to the securing mechanism 520 via other types of connection. Also, in the illustrated example, the securing mechanism 520 includes a top plate 530 for interfacing with a top side of the patient support 14, and a bottom plate 532 for interfacing with a bottom side of the patient support 14. The top plate 530 and the bottom plate 532 are configured to clamp against the patient support 14 to thereby secure the structure 504 relative to the patient support 14. A number of screws/bolts may be provided to secure the plates 530, 532 relative to the patient support 14. In other embodiments, the securing mechanism 520 may have other configurations, and may be configured to secure to the patient support 14 using other connection mechanisms.

During use of the apparatus 500, the light source 152 projects light onto the markers 502, and the cameras 154 sense the markers and generate corresponding image(s) of the markers 502. In some cases, the light provided by the light source 152 may be non-structured light in the infrared range (e.g., the light has wavelengths that are only in the infrared range). In such cases, the cameras 154 are configured to capture light in the infrared range that is reflected from the markers. In other embodiments, the light source 152 may provide light in other wavelengths that are different from the infrared wavelengths, and the cameras 154 may be configured to capture light having other wavelengths.

The processing unit 156 receives the images from the cameras 154, and processes the images to determine the position of the markers 502. For example, the processing unit 156 may determine the positions of the markers 502 as they appear in an image, and calculate a position of the frame 510 based on known relative positions of the markers 502 with respect to each other. The position of the frame 510 may include a location (e.g., X-coordinate, Y-coordinate, Z-coordinate, or any combination of the foregoing), and/or orientation (e.g., orientation about an x-axis, orientation about an y-axis, orientation about an z-axis, or any combination of the foregoing).

Also, because the position of the frame 510 relative to the patient support 14 is known (predetermined), by determining the position of the frame 510, the processing unit 156 can calculate the position of the patient support 14 based on the known relative positioning between the frame 510 and the patient support 14.

The position of the patient support 14 may be used for various proposes. For example, the position of the patient support 14 may be used by the processing unit 156 to track a position of the patient support 14 to make sure that the patient support 14 is at its intended position during the medical procedure. The processing unit 156 may also use the position of the patient support 14 to determine if the patient is too close to another component of the treatment system 10. The processing unit 156 may also use the position of the patient support 14 to confirm a position of the patient based on a known relative positioning between the patient and the patient support 14.

In other embodiments, the apparatus 500 may be attached to other components of the system 10. In some embodiments, there may be multiple apparatuses 500 for attachment to multiple components of the system 10. The camera(s) 154 is then used to capture images of the markers at the different apparatuses 500 for the purpose of monitoring positions of the different respective components. In some embodiments, each apparatus 500 may have a configuration for allowing the processing unit 156 processing the input image(s) from the camera(s) 154 to uniquely identify the apparatus 500. For example, a first apparatus 500 attached to the patient support 14 may have triangular shape markers, while a second apparatus 500 attached to a moving gantry may have circular shape markers. During use, the processing unit 156 processes the input image(s) from the camera(s) 154 to determine the positions of the respective components (e.g., patient support 14, gantry, etc.) based on the positions of the markers of the respective apparatuses 500. In some embodiments, the positions of the components may be used by the processing unit 156 to perform collision prevention. If the positions of the components indicate that they may be too close (e.g., if component-to-component distance between the positions is less than a certain prescribed threshold), and/or if the position of any of the components is too close to the patient (e.g., if component-to-patient distance is less than a certain prescribed threshold), then the processing unit 156 may determine that a collision may be imminent, and may generate a control signal to stop an operation of the system 10.

As shown in the above example, the apparatus 500 is advantageous in that it functions as a rigid body that provides a surrogate for a machine component. The rigid body may be stable when attached to the component. The rigid body may have a low profile and/or low mass for easy attachment to the component without creating a bulky configuration. The rigid body may be easily decoupled from the component, which provides for fast, accurate, and easy attachment anytime before, during, and/or after a medical procedure. The rigid body may also allow high accuracy tracking (e.g., sub-mm resolution tracking) of the component to which it is attached. The rigid body may also allow real-time tracking of the component to be performed with high accuracy (e.g., sub-mm accuracy).

In some embodiments, the apparatus 300, apparatus 500, and/or the optical system 150 may be configured to achieve certain optimization(s). For example, the apparatus 500, and/or the optical system 150 may be configured to have certain marker size, certain marker reflectivity, and/or certain spacing of the markers so that a desired positioning accuracy can be achieved based on optical viewing of the markers by the camera(s). Also, the apparatus 300 and/or the apparatus 500 may provide asymmetric target configuration to reduce (e.g., minimize) impact of target obscuration on accuracy. In addition, the optical system 150, the apparatus 300, and the apparatus 500 may be setup so that the view points of the camera(s) towards the apparatus 300 and the apparatus 500 is unique. This may also reduce geometric reconstruction errors.

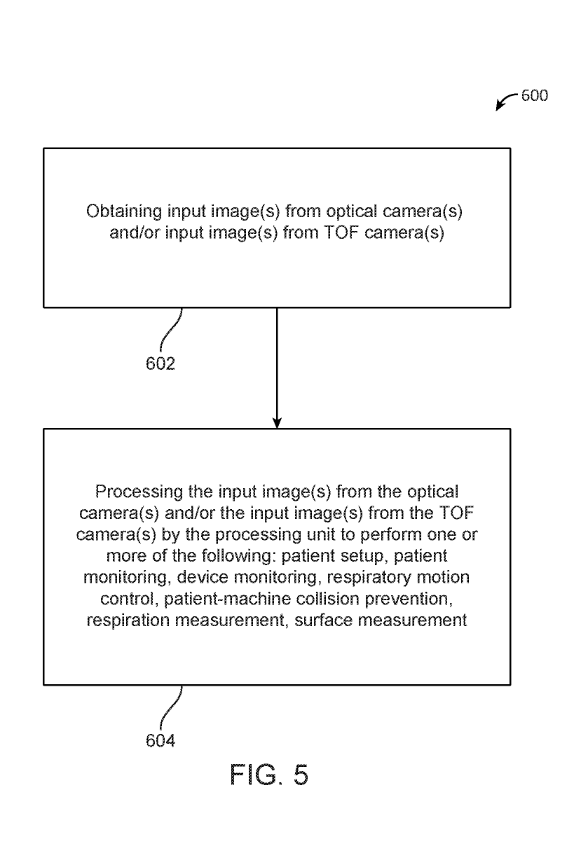

FIG. 5 illustrates a method 600 that may be performed by the optical system 150. First, input image(s) from optical camera(s) and/or input image(s) from TOF camera(s) is obtained (item 602). In some embodiments, the optical camera(s) may be the camera(s) 154, and the TOF camera(s) may be the TOF camera(s) 158. Also, in some embodiments, item 602 may be performed by the processing unit 156. Next, the input image(s) from the optical camera(s) and/or the input image(s) from the TOF camera(s) are processed by the processing unit to perform one or more of the following: patient setup, patient monitoring, device monitoring, respiratory motion control, patient-machine collision prevention, respiration measurement, and surface measurement (item 604). In some cases, the above features may be performed in a treatment room. Also, in some cases, the respiration measurement and surface measurement may be performed during a CT procedure.

In some embodiments, patient monitoring may be performed simultaneously or in an interleaved manner with device monitoring. In other embodiments, patient monitoring may be performed simultaneously or in an interleaved manner with patient-machine collision prevention. In further embodiments, device monitoring may be performed simultaneously or in an interleaved manner with patient-machine collision prevention. In still further embodiments, patient monitoring, device monitoring, and patient-machine collision prevention may be performed simultaneously or in an interleaved manner. In other embodiments, any combination of the features mentioned in item 604 may be performed simultaneously or in an interleaved manner. In some embodiments, the processing unit may include (1) a patient setup module configured to perform the patient setup, (2) a patient monitoring module configured to perform the patient monitoring, (3) a device monitoring module configured to perform the device monitoring, a respiratory motion controller configured to perform the respiratory motion control, and/or (4) a patient-machine collision prevention module configured to perform the patient-machine collision prevention. In some cases, the processing unit may also optionally include a device-device collision prevention module configured to perform device-to-device collision prevention. For example, the processing unit may be configured to monitor the moving gantry and the patient support to prevent collision between these two devices. As another example, the processing unit may be configured to monitor an imaging panel (e.g., a kV panel) and a position detection device (e.g., a Calypso console) to prevent collision between these two devices.

In some embodiments, items 602 and 604 are repeated during a medical procedure. Also, in some embodiments, the input image(s) from the optical camera(s) may be real-time image(s), and input image(s) from the TOF camera(s) may be real-time image(s). For example, the camera(s) 154 may be configured to repeatedly generate real-time input images, and the processing unit 156 processes these input images in real-time (e.g., within a short time after the image is generated, such as within 1 second, and more preferably within 0.5 second, and more preferably within 0.1 second) to monitor the position(s) of the component(s) of the system 10, and/or position of the patient.

It should be noted that the optical system 150 described herein is not limited to application with medical systems that provide radiation treatment beams, and that the optical system 150 may be employed in other types of medical system and procedures. For example, in other embodiments, the system 10 may be configured to provide proton beam, and the optical system 150 described herein may be used during a proton treatment procedure. As another example, the system 10 may be configured to provide imaging beam, and the optical system 150 described herein may be used during an imaging session. In some cases, the imaging may be performed using a CT machine, which may be a stationary machine, or a machine that is moveably coupled to a rail. Also, as used in this specification, the term “radiation” may include “proton beam”, a beam for treatment, or a beam for imaging.

In addition, in the above embodiments, the optical system 150 has been described with reference to it having reflective markers. In other embodiments, the optical system 150 may instead comprise actively illuminated markers. For example, active infrared (IR) markers may be used. In some embodiments, one or more active marker(s) may be configured to emit non-visible light. The active markers may be coupled to the patient, to a medical system, to part(s) of a room (e.g., to wall(s), floor, ceiling, etc.), or any combination of the foregoing. In such cases, the one or more camera(s) of the optical system 150 is configured to detect the non-visible light output from the active marker(s).

Also, it should be noted that as used in this specification, the term “image” is not limited to an image that is displayed, and may refer to an image that is not displayed (e.g., an image in data or digital form that is stored).

Specialized Processing System

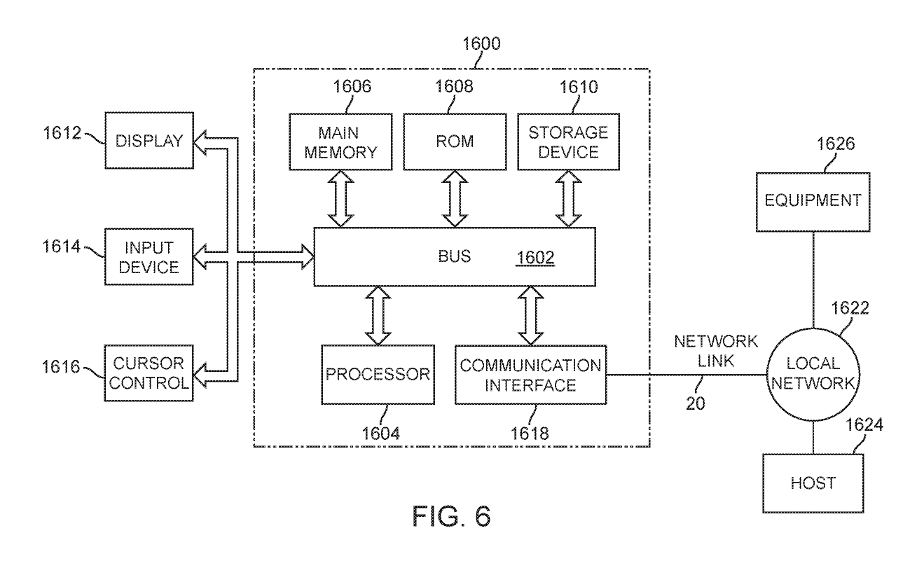

FIG. 6 is a block diagram illustrating an embodiment of a specialized processing system 1600 that can be used to implement various embodiments described herein. For example, the processing system 1600 may be configured to perform the method 600 of FIG. 5. The processing system 1600 may also be an example of the processing unit 156. The processing system 1600 may also be any processor described herein. Also, in some embodiments, the processing system 1600 may be configured to process real-time input images from the camera(s) 154 by comparing them with reference image, and/or real-time depth images from the TOF camera(s) 158 by comparing them with reference depth image.

Referring to FIG. 6, the processing system 1600 includes a bus 1602 or other communication mechanism for communicating information, and a processor 1604 coupled with the bus 1602 for processing information. The processor system 1600 also includes a main memory 1606, such as a random access memory (RAM) or other dynamic storage device, coupled to the bus 1602 for storing information and instructions to be executed by the processor 1604. The main memory 1606 also may be used for storing temporary variables or other intermediate information during execution of instructions to be executed by the processor 1604. The processor system 1600 further includes a read only memory (ROM) 1608 or other static storage device coupled to the bus 1602 for storing static information and instructions for the processor 1604. A data storage device 1610, such as a magnetic disk or optical disk, is provided and coupled to the bus 1602 for storing information and instructions.

The processor system 1600 may be coupled via the bus 1602 to a display 167, such as a cathode ray tube (CRT), for displaying information to a user. An input device 1614, including alphanumeric and other keys, is coupled to the bus 1602 for communicating information and command selections to processor 1604. Another type of user input device is cursor control 1616, such as a mouse, a trackball, or cursor direction keys for communicating direction information and command selections to processor 1604 and for controlling cursor movement on display 167. This input device typically has two degrees of freedom in two axes, a first axis (e.g., x) and a second axis (e.g., y), that allows the device to specify positions in a plane.

In some embodiments, the processor system 1600 can be used to perform various functions described herein. According to some embodiments, such use is provided by processor system 1600 in response to processor 1604 executing one or more sequences of one or more instructions contained in the main memory 1606. Those skilled in the art will know how to prepare such instructions based on the functions and methods described herein. Such instructions may be read into the main memory 1606 from another processor-readable medium, such as storage device 1610. Execution of the sequences of instructions contained in the main memory 1606 causes the processor 1604 to perform the process steps described herein. One or more processors (e.g., GPU(s)) in a multi-processing arrangement may also be employed to execute the sequences of instructions contained in the main memory 1606. In alternative embodiments, hard-wired circuitry may be used in place of or in combination with software instructions to implement the various embodiments described herein. Thus, embodiments are not limited to any specific combination of hardware circuitry and software.

The term “processor-readable medium” as used herein refers to any medium that participates in providing instructions to the processor 1604 for execution. Such a medium may take many forms, including but not limited to, non-volatile media, volatile media, and transmission media. Non-volatile media includes, for example, optical or magnetic disks, such as the storage device 1610. A non-volatile medium may be considered an example of non-transitory medium. Volatile media includes dynamic memory, such as the main memory 1606. A volatile medium may be considered an example of non-transitory medium. Transmission media includes coaxial cables, copper wire and fiber optics, including the wires that comprise the bus 1602. Transmission media can also take the form of acoustic or light waves, such as those generated during radio wave and infrared data communications.

Common forms of processor-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave as described hereinafter, or any other medium from which a processor can read.

Various forms of processor-readable media may be involved in carrying one or more sequences of one or more instructions to the processor 1604 for execution. For example, the instructions may initially be carried on a magnetic disk of a remote computer. The remote computer can load the instructions into its dynamic memory and send the instructions over a telephone line using a modem. A modem local to the processing system 1600 can receive the data on the telephone line and use an infrared transmitter to convert the data to an infrared signal. An infrared detector coupled to the bus 1602 can receive the data carried in the infrared signal and place the data on the bus 1602. The bus 1602 carries the data to the main memory 1606, from which the processor 1604 retrieves and executes the instructions. The instructions received by the main memory 1606 may optionally be stored on the storage device 1610 either before or after execution by the processor 1604.

The processing system 1600 also includes a communication interface 1618 coupled to the bus 1602. The communication interface 1618 provides a two-way data communication coupling to a network link 1620 that is connected to a local network 1622. For example, the communication interface 1618 may be an integrated services digital network (ISDN) card or a modem to provide a data communication connection to a corresponding type of telephone line. As another example, the communication interface 1618 may be a local area network (LAN) card to provide a data communication connection to a compatible LAN. Wireless links may also be implemented. In any such implementation, the communication interface 1618 sends and receives electrical, electromagnetic or optical signals that carry data streams representing various types of information.

The network link 1620 typically provides data communication through one or more networks to other devices. For example, the network link 1620 may provide a connection through local network 1622 to a host computer 1624 or to equipment 1626 such as a radiation beam source or a switch operatively coupled to a radiation beam source. The data streams transported over the network link 1620 can comprise electrical, electromagnetic or optical signals. The signals through the various networks and the signals on the network link 1620 and through the communication interface 1618, which carry data to and from the processing system 1600, are exemplary forms of carrier waves transporting the information. The processing system 1600 can send messages and receive data, including program code, through the network(s), the network link 1620, and the communication interface 1618.

Although particular features have been shown and described, it will be understood that they are not intended to limit the claimed invention, and it will be made obvious to those skilled in the art that various changes and modifications may be made without departing from the spirit and scope of the claimed invention. The specification and drawings are, accordingly to be regarded in an illustrative rather than restrictive sense. The claimed invention is intended to cover all alternatives, modifications and equivalents.

What is claimed:

1. An apparatus for use in a medical procedure, comprising:
a structure for coupling to a patient;
a plurality of reflective markers coupled to the structure, wherein the reflective markers are configured to reflect non-visible light; and
a securing mechanism configured to secure the structure relative to the patient.

2. The apparatus of claim 1, wherein the structure comprises a frame, and a first base coupled to the frame.

3. The apparatus of claim 2, wherein the securing mechanism comprise a first adhesive for attachment to the patient.

4. The apparatus of claim 2, further comprising a nose piece coupled to the frame.

5. The apparatus of claim 4, wherein the nose piece comprises an adhesive for attachment to the patient.

6. The apparatus of claim 2, wherein the frame comprises a laser alignment mark.

7. The apparatus of claim 1, wherein the plurality of reflective markers comprises four reflective markers.

8. The apparatus of claim 2, further comprising a second base having a second adhesive for attachment to the patient, wherein the second base is coupled to the frame.

9. The apparatus of claim 2, wherein the first base is rotatably coupled to the frame.

10. The apparatus of claim 9, wherein the first base is rotatably coupled to the frame via a ball and socket connector.

11. The apparatus of claim 2, wherein the first base is configured to detachably couple to the frame.

12. The apparatus of claim 2, wherein the first base is configured to detachably couple to the frame via a snap-connector.

13. The apparatus of claim 1, wherein the structure is configured for coupling to a head of the patient.

14. The apparatus of claim 1, wherein the structure is configured for coupling to a torso of the patient.

15. The apparatus of claim 1, wherein the structure is configured for coupling to a limb of the patient.

16. The apparatus of claim 1, wherein the structure comprises a frame and the securing mechanism comprises a face mask coupled to the frame.

17. The apparatus of claim 1, wherein the structure is configured for coupling to a skin above an ear of the patient.

18. The apparatus of claim 1, wherein the structure comprises eyewear.

19. The apparatus of claim 1, wherein the structure comprises a strap for placement around a head of the patient.

20. The apparatus of claim 19, wherein the securing mechanism comprises a strap connector or a strap tightening knob.

21. The apparatus of claim 1, wherein the structure comprises a mouth piece.

22. The apparatus of claim 1, wherein the structure comprises a hat.

23. The apparatus of claim 22, wherein the hat comprises a first portion made from a first material, and a second portion made from a second material, and wherein the first material is more elastic than the second material.

24. The apparatus of claim 1, further comprising one or more cameras for viewing the plurality of reflective markers, and one or more light sources for providing structured light for surface scanning.

25. An apparatus for use in a medical procedure, comprising:
a structure for coupling to a component of a medical system;
a plurality of reflective markers coupled to the structure, wherein the reflective markers are configured to reflect non-visible light; and
a securing mechanism configured to secure the structure relative to the patient support.

26. The apparatus of claim 25, wherein the structure comprises a first arm, and a second arm moveably coupled to the first arm.

27. An apparatus for use in a medical procedure, comprising:
a first light source configured to project a first structured light onto an object, wherein the first structured light is non-visible;
a first camera configured to obtain a first image of the first structured light as projected onto the object;
a second camera configured to obtain a second image of the first structured light as projected onto the object; and
a processing unit configured to process the first and second images from the first camera and the second camera;
wherein the first camera and the second camera are configured for non-visible light detection.

28. The apparatus of claim 27, further comprising a first time-of-flight camera configured to obtain a first depth image of the object.

29. The apparatus of claim 28, further comprising a second time-of-flight camera configured to obtain a second depth image of the object.

30. The apparatus of claim 27, further comprising a frame, wherein the first light source, the first camera, and the second camera are coupled to the frame.

31. The apparatus of claim 30, wherein the first camera is moveably mounted to the frame, and the second camera is moveably mounted to the frame.

32. The apparatus of claim 27, wherein the processing unit is configured to perform imaging processing based on input from the first camera and the second camera for patient setup, patient monitoring, device monitoring, collision prevention, respiratory motion control, or any combination of the foregoing.

33. The apparatus of claim 27, wherein the processing unit is configured to determine a surface based on input from the first camera and the second camera.

34. The apparatus of claim 33, wherein the surface corresponds with an abdominal region of a patient, and/or a chest of the patient, and wherein the processing unit is further configured to correlate a position of the surface with an interior region of the patient.
